(19) United States
(12) Patent Application Publication
(10) Pub. No.: US 2016/0136338 A1
(43) Pub. Date: May 19, 2016

(54) AUTOMATIC EXCREMENT PROCESSING DEVICE

(71) Applicant: ANGELWINGS, INC., Pyeongtaek (KR)

(72) Inventors: Joon-Woo LEE, Pyeongtaek (KR); Eun-Kyung LEE, Pyeongtaek (KR)

(21) Appl. No.: 14/901,337

(22) PCT Filed: Jun. 25, 2014
(86) PCT No.: PCT/KR2014/005650

§ 371 Date: Dec. 28, 2015

(30) Foreign Application Priority Data
Jun. 26, 2013 (KR) 10-2013-0073810
Sep. 3, 2013 (KR) PCT/KR2013/007938

(51) Int. Cl. A61M 1/00; A61F 5/441; A61F 5/442; A47K 10/00; A61F 5/455; A61M 3/06; A47K 3/26; A61F 5/44; A61F 5/453

(52) U.S. Cl. CPC A61M 1/0031; A61F 5/4405; A61F 5/441; A61F 5/442; A61F 5/453; A61F 5/455; A61M 3/06; A47K 3/26; A47K 10/00

(57) ABSTRACT

The present invention relates to an automatic excrement processing device comprising: a cup unit (10) worn on a portion of a user at which the excretory organs are located, and forming a space (11) in which urine is suctioned that is partitioned from a space (13) in which feces is suctioned; an excrement processing main body (60) connected to the cup unit (10) and suctioning excrement discharged into the cup unit (10) and storing same; a sensor unit provided on the cup unit (10), connected to the excrement processing main body (60), and sensing excrement; a washing nozzle unit (21) performing a washing function; a suctioning nozzle unit (49) suctioning excrement; and a drying nozzle unit (31) performing a drying function. The present invention has the advantages that the device provides a good feeling when worn, and wash excrement processing is possible through accurate sensing of excrement.

### TECHNICAL FIELD

The present invention relates to an automatic excrement processing device, and in particular to an automatic excrement processing device which may provide a wash and comfort excrement process.

### BACKGROUND ART

In recent years, as our society enters an elderly society stage, the population of elderly citizen fast increases, and patients who need recuperation and are suffering from behavior also increase.

In case of patients who need recuperation and are suffering from behavior, they cannot control their bodies for themselves, whereupon they need helps from nearby others. Since they are unable to go to bathrooms, they need to wear diapers. In case that they excavate excrement, guardian or caregiver must remove excrements.

If the diapers are used, it inconveniently needs to change the diaper whenever the user excavates excrement. If the user does not take a shower immediately after excavating excrement, the user may not feel washed, thus causing inconvenience when in use.

Since the excavation of excrement might be a sensitive matter which may cause the user to feel shy and sham, the patients as well as guardians may receive spiritual, physical and economical burdens.

The prior art related with the present invention is KR1020080000941 A.

### DISCLOSURE OF INVENTION

### Technical Problem

Accordingly, it is an object of the present invention to provide an automatic excrement processing device which is able to provide a good wearing comfort while allowing a user to easily move, and a comfort excrement process is available even when a user lies on his back in a normal posture or lies on his side.

It is another object of the present invention to provide an automatic excrement processing device wherein excrement may be accurately recognized after a user excavates excrement, and an excrement suction, washing and bidet function can be automatically carried out, which may allow a user to cleanly perform an excrement process. Noise is low, and the excrement does not flow reversely or does not leak to the outside, and the smell of excrement does not leak to the outside as well.

It is further another object of the present invention to provide an automatic excrement processing device wherein it is easy to engage or disengage a cup unit and an excrement processing main body, and a connection pipe which connects the cup unit and the excrement processing main body, and the smell of excrement does not flow reversely to the cup unit, and the present invention can apply to various body types of users.

### Solution to Problem

To achieve the above objects, there is provided an automatic excrement processing device which may include, but is not limited to, a cup unit which is worn at a portion corresponding to a user's excretory organ and is divided into a space for sucking urine and a space for sucking feces; an excrement processing main body which is connected with the cup unit and is configured to suck and store the excrement which is excavated into the cup unit; a sensor unit which is provided in the cup unit and is connected with the excrement processing main body and is able to detect the excrement; a washing nozzle unit which is configured to carry out a washing function; a suction nozzle unit which is able to suck the excrement; and a drying nozzle unit which is able to carry out a drying function, wherein when the user excavates excrement, the excrement can be automatically detected, and a washing, suction, bidet and drying can be carried out.

### Advantageous Effects

The cup unit according to the present invention may be configured to match with various body types in such a way to provide a wrinkle unit, and a cup unit main body may be made of an ABS material, and a portion contacting with user's hips is covered with a silicon material so as to distribute the pressure when a user sits.

To this end, the cup unit of the present invention may allow to obtain a close contact with a user's excretory organ, thus obtaining a good wearing comfort and preventing any pressure sore of a user while preventing an excrement leaking problem. Any deformation of a cup unit due to a user's pressing may be prevented, whereupon a sensing power of a sensor unit can be enhanced, thus obtaining a wash processing of excrement.

In addition, the cup unit of the present invention may be configured in such a way that electric sensors are arranged on an edge thereof, so it is possible to accurately detect urine even when a user lies on his side, and the interior of the cup unit can be effectively configured narrow and efficient in terms of suction.

In the present invention, a washing effect on anus can be enhanced in case of excrement since an anus nozzle unit can operate on front and rear sides. Since a cleaning and washing nozzle can strongly push excrement in the direction of a suction nozzle unit, the excrement can be processed wash, and a space into which excrement is sucked can be maintained always wash.

In the present invention, a suction nozzle unit which is configured to transfer excrement is singularly connected with an excrement processing main body in a connection pipe, and a washing nozzle unit, a drying nozzle and a sensor unit are together connected through a coupling to the excrement processing main body. For this reason, the cup unit and the connection pipe, and the connection pipe and the excrement processing main body can be easily engaged or disengaged, and washing, movement and storage are easy.

In the present invention, when excavating excrement, suction, washing, cleaning and drying of excrement can be automatically performed. The device of the present invention may be set based on a user's preference in such a way to control water pressure, drying temperature and washing temperature which may be divided into multiple stages, thus carrying out a clean excrement process.

### BRIEF DESCRIPTION OF DRAWINGS

FIG. 1 is a view illustrating an automatic excrement processing device according to an exemplary embodiment of the present invention.

FIG. 2 is a view illustrating a front side of a cup unit according to an exemplary embodiment of the present invention.

FIG. 3 is a side view illustrating an inner configuration of a cup unit according to an exemplary embodiment of the present invention.

FIGS. 4 and 5 are views illustrating a configuration wherein a close contact unit is engaged to a cup unit according to the present invention.

FIG. 6 is a view illustrating a backside of a cup unit according to an exemplary embodiment of the present invention.

FIG. 7 is a view illustrating an excrement processing main body according to an exemplary embodiment of the present invention.

FIG. 8 is a view illustrating a position where a suction tank and a washing water storing tank are arranged on an excrement processing main body according to an exemplary embodiment of the present invention.

FIG. 9A is a view illustrating an upper surface of a suction tank according to an exemplary embodiment of the present invention, and FIG. 9B is a view when viewing the configuration in FIG. 9A in the direction A-A′, and FIG. 9C is a view when viewing the configuration in FIG. 9A in the direction B.

FIG. 10 is a view illustrating a display unit according to an exemplar embodiment of the present invention.

FIG. 11 is a view for describing an operation principle of an automatic excrement processing device according to the present invention.

FIG. 12 is a view illustrating a backside of a cup unit according to another exemplary embodiment of the present invention.

FIG. 13 is a view illustrating a backside of an excrement processing main body according to another exemplary embodiment of the present invention.

FIG. 14 is a view for describing an operation principle of an automatic excrement processing device according to another exemplary embodiment of the present invention.

FIGS. 15 to 17 are views illustrating a cup unit according to further another exemplary embodiment of the present invention.

* Legend of reference numbers of key components*

| | |
|---|---|
| 10: cup unit | 11: Urine sucking space |
| 13: Feces sucking space | A: Upper cup |
| B: Lower cup | 15: Wrinkle unit |
| 17: Partition unit | 19: Communication hole |
| 21: Washing nozzle unit | 23: Men's washing nozzle |
| 25: Women's washing nozzle | 27, 28: Anus washing nozzle |
| 29: Washing and cleaning | 31: Drying nozzle unit |
| nozzle | |
| 33: Close contact unit | 33a: First close contact unit |
| 33b: Second close contact unit | 35: Concave slit |
| 37: Insertion protrusion | 39: Wearing detection sensor |
| 41: Electric sensor | 43: Photo sensor |
| 45: Air hole | 47: Discharge passage |
| 48: Coupling unit | 49: Suction nozzle unit |
| 50: Connection pipe | 51: Support unit |
| 53: Protection cover | 60: Excrement processing main body |
| 61: Case | 63: Suction tank-arranged space |
| 65: Storing tank-arranged space | 67: Suction unit-arranged space |
| 68: Coupling unit engaging unit | 69: Excrement collection port |
| 71a, 71b: Noncontact sensor | 73: Upper cover |
| 75: Suction tank | 77: Space unit |
| 79: Excrement inlet port | 83: Partition unit |
| 85: Air discharge pipe | 87: Main cover |
| 89: Assistant cover | 91: Handle |
| 93: Washing water storing tank | 95: Water pump |
| 97: Suction unit | 99: Suction motor |
| 101: Instant heater | 103: Supply amount control unit |
| 105: Assistant pump | 107: Control unit |
| 109: Display unit | 111: Moving wheel |
| 113: Handle unit | 115: Power plug |

### BEST MODES FOR CARRYING OUT THE INVENTION

The exemplary embodiments of the present invention will be described in detail with reference to the accompanying drawings.

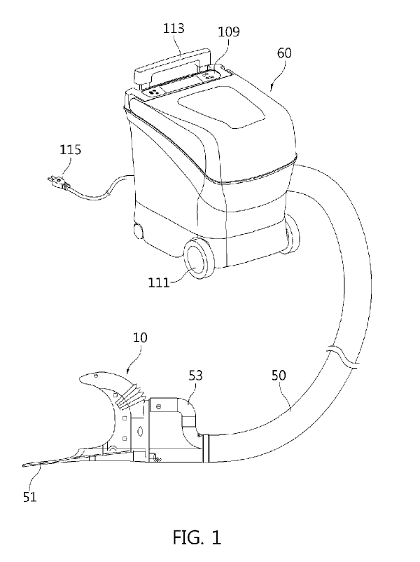

As illustrated in FIGS. 1 to 3, the automatic excrement processing device according to the present invention may include, but is not limited to, a cup unit 10 which is worn at a portion corresponding to an excretory organ of a user and the inside of which is divided into a urine sucking space 11 and a feces sucking space 13; and an excrement processing main body 60 which is connected with the cup unit 10 and is configured to suck and store the excrement excavated into the cup unit 10.

In the present invention, there are provided a sensor unit which is provided at the cup unit 10 and is connected with the excrement processing main body 60 and is able to detect excrement; a washing nozzle unit 21 for carrying out a washing function; and a drying nozzle unit 31 for carrying out a drying function. When the user excavates excrement into the cup unit 10, the excrement can be automatically detected, and a washing, sucking, bidet and drying are carried out.

The cup unit 10 is worn at a portion where the user's excretory organ position. The cup unit 10 is able to carry out a washing function at the user's excretory organ at the moment the cup unit 10 sucks excrement after the user has defecated, thus easily carrying out defecation. When the user urinates and defecates, the cup unit 10 may carry out the suctions of excrement mixed with urine and feces and may carry out the washing and drying function with respect to the excretory organ.

The excretory organ represents a genital organ portion through which urine discharges and an anus portion through which feces discharges. The user represents a patient who needs recuperation or a patient or elderly person who suffers from behavior. The excrement represents user's urine and feces.

The cup unit 10 may be formed of an upper cup “A” where a urine sucking space 11 is formed, and a lower cup “B” wherein a feces sucking space 13 is formed.

The cup unit 10 is configured in such a way that its front side is formed in a C-shaped curve, and a wrinkle unit 15 is formed at its rear side in order for the upper cup “A” to be tilted with respect to the lower cup “B”. The cup unit 10 is worn in such a way that its front side corresponds to a portion where the user's excretory organ positions.

The wrinkle unit 15 may be configured to allow the upper cup “A” to be tilted 90° with respect to the lower cup “B”. The wrinkle unit 15 may allow for an easier wearing of the cup unit 10 by facilitating the top of the cup unit 10 to smoothly tilt. In addition, the wrinkle unit 15 may allow the front side of the cup unit 10 to contact close with the portion where the user's excretory organ positions with the aid of recovery force when the cup unit 10 is worn at the user's excretory organ portion.

The wrinkle unit 15 may be configured to have as many as wrinkles to match with various body types, for example, Asian, Westerner, etc. For example, for an ordinary body type user who has a weight of 40 kg to 70 kg, it is preferred to manufacture a wrinkle unit having 5 to 6 wrinkles.

The urine sucking space 11 and the feces sucking space 13 may be partitioned by the partition unit 17. The partition unit 17 may include a communication hole 19 which may allow to communicate the urine sucking space 11 and the feces sucking space 13. The communication hole 19 may allow the urine discharged into the urine sucking space 11 to be sucked into the suction nozzle unit 49 through the feces sucking space 13.

If the urine sucking space 11 and the feces sucking space 13 are partitioned by the partition unit 17, it may be possible to prevent any disease, for example, a secondary infection, which may occur since feces may be mixed near the genital organ portion of the user when defecating. In case of a male user, such a partition unit 17 may allow to prevent the genital organ portion from being sucked into the suction nozzle unit 49 by suction force which was originally intended to be used to suck excrement.

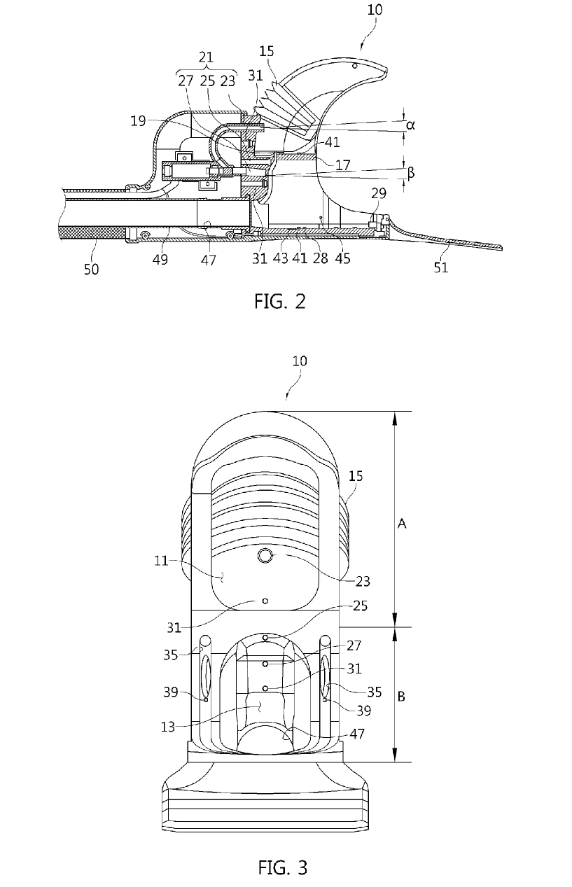

As illustrated in FIGS. 2 and 3, the cup unit 10 may include a washing nozzle unit 21 which is able to wash a user's excretory organ. The washing nozzle unit 21 may include a men's washing nozzle 23, a women's washing nozzle 25, anus washing nozzles 27 and 28, and a cleaning and washing nozzle 29.

An end of the men's washing nozzle 23 is exposed into the urine sucking space 11, thus washing the genital organ portion of the male user, and the men's washing nozzle 23 may be configured to spray washing water from inside to outside.

An end of the women's washing nozzle 25 is exposed into the feces sucking space 13, thus washing the genital organ portion of the female user. In case of women, since the genital organ portion and the anus portion position close to each other, it is preferred that the end of the women's washing nozzle 25 is exposed into the feces sucking space.

If it needs to adjust the ratio of the urine sucking space 11 and the feces sucking space 13, the device may be configured in such a way that the end of the women's washing nozzle 25 is exposed into the urine sucking space 11.

In the present exemplary embodiment of the present invention, since the cup unit 10 is configured for both women and men, the cup unit 10 may equip with both the men's washing nozzle 23 and the women's washing nozzle 25. If the cup unit 10 is manufactured separate for the uses of women and men, the men's cup unit equips with the men's washing nozzle 23, and the women's cup unit equips with the women's washing nozzle 25.

Since the women's washing nozzle 25 is provided for the sake of a bidet function, it is preferred that the women's washing nozzle 25 uses a type wherein washing water falls from top to bottom.

Ends of the anus washing nozzles 27 and 28 are exposed into the feces sucking space 13, thus washing the anus portion of the user in the forward and backward directions.

There are two anus washing nozzles 27 and 28 wherein an end of one of the two anus washing nozzles 27 and 28 is exposed into the urine sucking space 13, and the other one of the two anus washing nozzles 27 and 28 is exposed into the feces sucking space 13, the other one of the two anus washing nozzles 27 and 28 being disposed on the bottom surface of the cup unit 10. The above two anus washing nozzles 27 and 28 are able to enhance an anus washing power since the anus washing can be carried out in the forward and backward directions just after the defecation has been made.

It is preferred that the anus washing nozzles 27 and 28 are configured to spray washing water from inside to outside for the sake of easier anus washing.

The installation angles of the men's washing nozzle 23, the women's washing nozzle 25 and the anus washing nozzle 27 may be adjusted within a set range. For example, the men's washing nozzle 23 and the women's washing nozzle 25 may be adjusted within an angle range of about 2°, and the anus washing nozzles 27 and 28 may be adjusted within an angle range of about 5°.

The men's washing nozzle 23, the women's washing nozzle 25 and the anus washing nozzle 27 may be movable forward and backward within a predetermined range. The forward and backward movements of the men's washing nozzle 23, the women's washing nozzle 25 and the anus washing nozzle 27 may be carried out by a user's operation on the display unit which will be described later.

The cleaning and washing nozzle 29 is configured to spray washing water toward the bottom surface of the feces sucking space 13, thus pushing the excrement in the direction of the suction nozzle unit 49. The cleaning and washing nozzle 29 is installed passing through the bottom surface of the cup unit 10 and an end of the cleaning and washing nozzle 29 is open in the direction being opposite to the suction nozzle unit 49. The cleaning and washing nozzle 29 sprays washing water in the direction of the bottom surface of the feces sucking space 13, whereupon the feces can be easily sucked into the suction nozzle unit 49, and the feces sucking space 13 can be cleaned wash.

Different from the men's washing nozzle 23, the women's washing nozzle 25 and the anus washing nozzles 27 and 28, the cleaning and washing nozzle 29 can receive washing water with the aid of the operation of the assistant pump 105. Different from the water pump 95 which is able to supply washing water to at least one or more of the washing nozzles 23, 25 and 27, the assistant pump 105 supplies washing water to one cleaning and washing nozzle 29, with the same capacity, it is possible to easily push the excrement in the direction of the suction nozzle unit 49 in such a way to increase the water pressure of the washing water which is sprayed from the cleaning and washing nozzle 29.

In the present exemplary embodiment of the present invention, an example has been described, wherein there are provided five components, more specifically, one men's washing nozzle 23, one women's washing nozzle 25, two anus washing nozzles 27 and 28, and one cleaning and washing nozzle 29, but such a configuration is not limited thereto. Five or more than five components may be proved for the sake of washer management of the cup unit 10.

The suction nozzle unit 49 may be connected to the discharge passage 47 formed at the cup unit 10. The discharge passage 47 may be formed on the backside of the cup unit 10 and may communicate with the feces sucking space 13. The suction nozzle unit 49 is configured to suck increment and discharge to the increment processing main body 60.

The drying nozzle unit 31 is exposed in the feces sucking space 13 and is able to spray air (wind) so as to dry the excretory organ.

The air discharged into the feces sucking space 13 through the drying nozzle unit 31 is inputted into the backside of the cup unit 10 and may be sprayed into the feces sucking space 13 through the cleaning and washing nozzle 29. If the washing water is sprayed together with air from the cleaning and washing nozzle 29, the water pressure of the washing water can be increased, thus enhancing the cleaning power of the excrement.

The cup unit 10 is made of an ABS material, and a portion which contacts with a user is covered with a silicon material.

If the cup unit 10 is made of an ABS material, it is possible to prevent any error operation of the sensor unit which might occur when the cup unit is pressed when the user wears the cup unit 10.

If the cup unit 10 is made of a silicon material, a wearing comfort is good thanks to flexibility and stickiness, but error operation might occur in the sensor unit due to any deformation when the silicon material is pressed. To this end, the cup unit 10 is made of an ABS material so as to enhance a detection performance of the sensor unit, and a portion which contacts with a user in the cup unit 10 is covered with a silicon material, thus enhancing a wearing comfort.

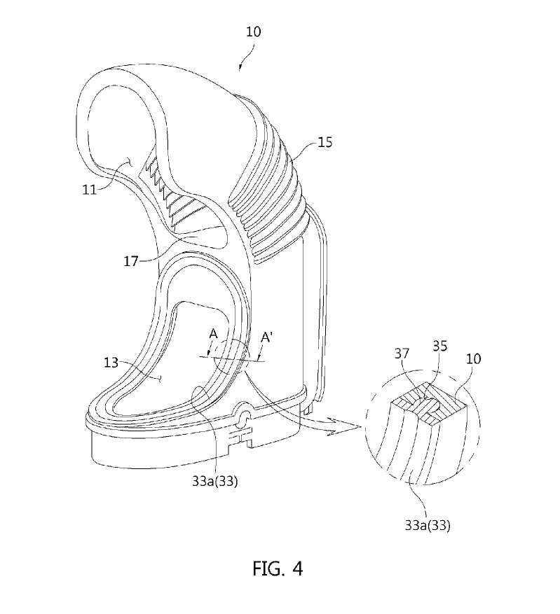

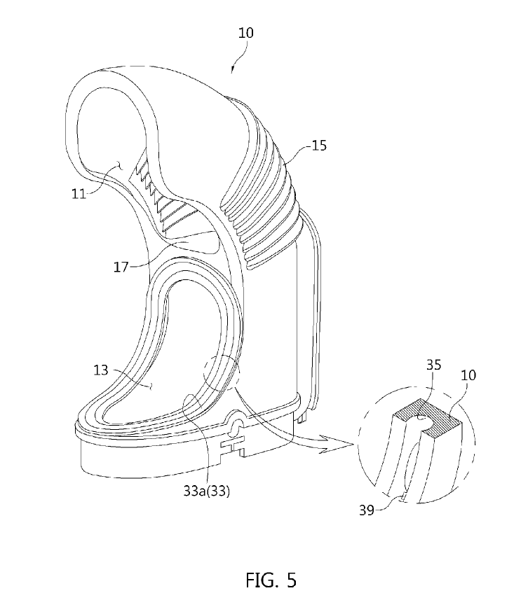

For example, as illustrated in FIGS. 4 and 5, the cup unit 10 may include a close contact unit 33 which is made of a silicon material and contacts close with hips where an anus of the user's excretory organ positions.

The close contact unit 33 may include an insertion protrusion 37 which is fit into the concave slit 35 formed at the cup unit 10, whereupon engagement may be carried out in such a way that the insertion protrusion 37 of the close contact unit 33 is fit into the concave slit 35 of the cup unit 10. The close contact unit 33 may define a mouth rim of the feces sucking space 13, whereupon the user's hips may contact close, and a good wearing comfort can be obtained by distributing pressing pressure.

As not illustrated in the drawing, an elastic member may be provided between the concave slit 35 and the insertion protrusion 37. The elastic member can provide elasticity at a portion with which the user's hips contacts close, so the user's hips can contact even closer with the cup unit 10. Here, the elastic member may be formed of an ordinary coil spring.

Since the elastic member is movable within a predetermined angle range in upward, downward, leftward and rightward directions with respect to the close contact unit 33, the cup unit 10 can remain worn in place by the user even when the user changes posture.

The close contact unit 33 may be variously sized based on the sizes of the user's hips.

For example, as illustrated in FIG. 4, a first close contact unit 33a configured to narrow the mouth of the feces sucking space 13 may be engaged to the cup unit 10 or otherwise as illustrated in FIG. 5, a second close contact unit 33b configured to maximally widen the mouth of the feces sucking space 13 may be engaged to the cup unit 10. The first close contact unit 33a and the second close contact unit 33b may be exchanged and used based on the user's hip sizes.

The cup unit 10 may include a sensor unit. The sensor unit may include, but is not limited to, a wearing detection sensor 39, an electric sensor 41, and a photo sensor 43.

The wearing detection sensor 39 is installed at the cup unit 10 and is configured to detect whether or not the user's hips contact close with the cup unit 10. The wearing detection sensor 39 may allow the washing water to be supplied to the cup unit 10 only when the user's hips contact close with the close contact unit 33. The wearing detection sensor 39 may be formed of a pressure sensor.

The wearing detection sensor 39 may be disposed where the close contact unit 33 engages in the cup unit 10 or may be disposed at the side of a suction unit 97 which will be described later. In the present exemplar embodiment of the present invention, as illustrated in FIG. 5, it is disposed at the concave slit 35 of the cup unit 10.

The electric sensor 41 may be installed at an edge of the bottom surface of the cup unit 10, thus detecting urine and feces. The electric sensor 41 may selectively carry out a suction, washing, and bidet function based on urine and feces. The electric sensor 41 is able to detect whether or not the excrement that the user has excavated in the cup unit is urine or feces based on a resistance value in such a way to flow resistance.

As illustrated in FIG. 2, the electric sensor 41 is disposed at the upper cup “A” and the lower cup “B” and is able to detect if the excrement excavated in the urine sucking space 11 and the feces sucking space 13 is urine or feces.

The electric sensor 41 is preferably installed multiple in number on an edge of the bottom surface of the cup unit 10. If the electric sensor 41 is installed multiple in number on an edge of the bottom surface of the cup unit 10, it is possible to detect urine even though a user lies on the ground, and the inside of the cup unit 10 may be configured narrow and may be configured for the sake of smooth suction. If the inside of the cup unit 10 is configured narrow, the width of the cup unit 10 can be made small, which may result in a good wearing comfort.

The photo sensor 43 is installed on the bottom surface of the cup unit 10 and is able to detect feces. It is preferred that the photo sensor 43 is installed on the bottom surface of the cup unit 10 so as to detect feces. The photo sensor 43 is positioned on the bottom surface of the cup in such a way to be integral with a PCB which has been manufactured sealed, thus improving a feces detection power.

It is possible to detect if the excrement is feces or urine using the electric sensor 41, but the electric sensor 41 is more appropriate to recognize urine, so there may any error. For the sake of accurate recognition of feces, a photo sensor is used. The photo sensor 43 is able to emit light into the feces sucking space 13 so as to detect any movement in feces or temperature, thus detecting the presence of feces.

The cup unit 10 may include an air hole 45 so as to prevent any formation of vacuum in the urine sucking space 11 and the feces sucking space 13. The air hole 45 may be configured to receive air through the rear end of the cup unit 10 and may allow to form an air flow passage at its side surface. The air hole 45 is provided so as to prevent any formation of vacuum in the feces sucking space 13 which formation may occur due to the air pressure used to suck feces even though the user's hips contact close with the close contact unit 33.

If the feces sucking space 13 becomes a vacuum state, hernia may occur due to the air pressure used to suck feces. It is preferred that the air hole 45 is formed at the cup unit 10 and is sized small enough not to have any effect on the suction of feces.

As not illustrated in the drawings, an air discharge hole may be further provided so as to spray air into the feces sucking space 13. The air discharge hole is open in the direction of the discharge passage 47 of the cup unit 10, whereupon the excrement can be pushed toward the suction nozzle unit 49 when sucking the excrement, thus obtaining an easier suction of excrement through the suction nozzle unit 49.

It is preferred that the air discharge hole is configured based on a principle wherein external air can be sucked inward and sprayed with the aid of air pressure used to suck excrement. For example, an air inlet hole is formed open to the rear end of the cup unit 10, and an air discharge hole may be formed open to the feces sucking space 13 while communicating with the air inlet hole through an empty space of the concave slit 35 to which the close contact unit 33 is engaged. In this case, the external air inputted into the air inlet hole can pass through the empty space of the concave slit 35 and then is collected in the air discharge hole and is discharged into the suction nozzle unit 49, whereupon the force for pushing the excrement toad the suction nozzle unit 49 can be increased.

In addition, the air discharged through a suction motor 99, which will be described later, can be inputted into the rear end of the cup unit 10 and can be sprayed from the cleaning and washing nozzle 29.

The air discharged into the feces sucking space 13 through the drying nozzle unit 31 instead of the air discharge hole can be inputted into the rear surface of the cup unit 10 and can be sprayed into the feces sucking space 13 through the cleaning and washing nozzle 29, whereupon the force that the washing water pushes excrement toward the suction nozzle unit 49 can be increased.

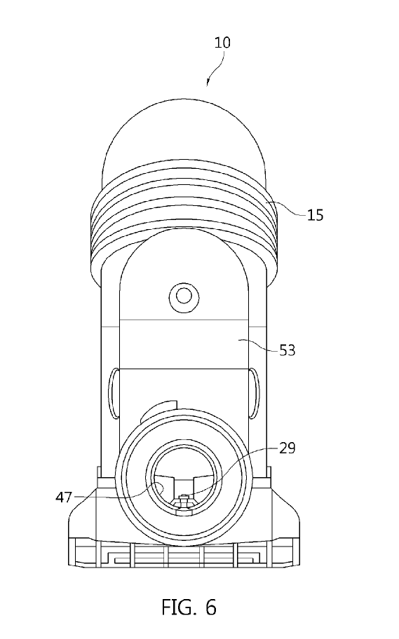

As illustrated in FIGS. 2 and 6, the cup unit 10 may include a discharge passage 47 communicating with the feces sucking space 13, and to the discharge passage 47, the suction nozzle unit 49 may be connected so as to suck excrement and transfer to the excrement processing main body 60.

The suction nozzle unit 49 is covered together with various nozzles and electric cables by the connection pipe 50 and is connected to the excrement processing main body 60. Various nozzles may be formed of a nozzle for supplying washing water and a nozzle for supplying wind to the drying nozzle unit 31. Electric cables may include components used to electrically connect the cup unit 10 and the excrement processing main body 60 for the sake of operations of the cup unit 10.

Nozzles for supplying washing water and nozzles for supplying drying wind are connected to the backside of the cup unit 10, whereupon it needs to make the exterior look good by installing a protection cover 53.

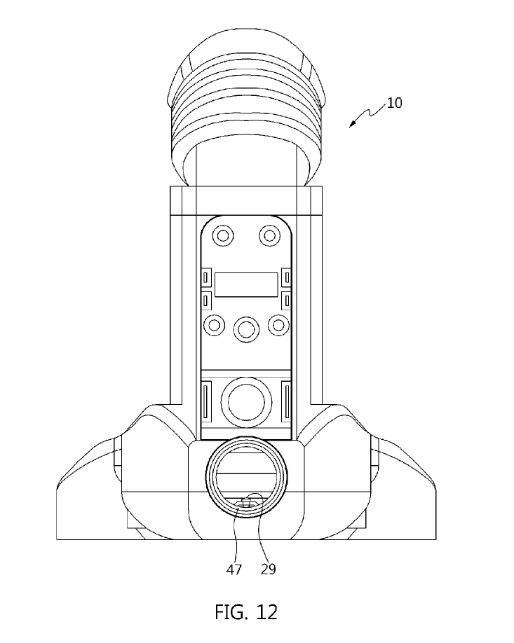

According to another exemplary embodiment of the present invention, as illustrated in FIGS. 2 and 12, the cup unit 10 is connected to the excrement processing main body 60 and the connection pipe 50, and the suction nozzle unit 49 in the connection pipe 50 is singularly connected to the excrement processing main body 60, and the washing nozzle unit 21, the drying nozzle unit 31 and the sensor unit are connected to the excrement processing main body 60 through the coupling unit 48 which is provided together, thus obtaining an easier engaging and disengaging of the cup unit 10 and the connection pipe 50, and the connection pipe 50 and the excrement processing main body 60.

If it is easy to engage or disengage the cup unit 10 and the connection pipe 50, the connection pipe 50 and the excrement processing main body 60, the cleaning and movement and storage of the cup unit 10 and the washing nozzle unit 21, the drying nozzle unit 31, and the suction nozzle unit 49 become easier.

The suction nozzle unit 49 may include an automatic valve which is opened when any excrement in the cup unit 10 is detected. The automatic valve is able to prevent the smell of the suction tank 75 from reversely flowing to the cup unit 10 through the suction nozzle unit 49. It is preferred that the automatic valve is provided at the suction nozzle unit 49 corresponding to the position near the excrement processing main body 60.

It is preferred that the bottom of the cup unit 10 is formed circular for the sake of easier posture change in case of a bed-ridden patient.

On the bottom surface of the cup unit 10, a support unit 51 is provided so as to support the cup unit 10. The cup unit 10 may be separable from the support unit 51 for the sake of easier washing. The support unit 51 is formed long in length, thus stably supporting the cup unit 10.

It is preferred that the cup unit 10 is designed to have a width of 14˜17 cm and a height of 15˜25 cm in consideration of a user's body type. It is also preferred that the support unit 51 is designed to have a length of 15˜28 cm, and the portion which protrudes toward the front side of the cup unit 10 has a gradually thinning thickness, thus stably supporting the bottom surface of the cup unit 10.

A ultraviolet ray lamp member (not shown) is provided, which is able to emit ultraviolet ray to the cup unit 10. The ultraviolet ray lamp member may be provided in a form of a device separate from the cup unit.

The excrement processing main body 60 is provided for the sake of storage and washing of the excrement.

The excrement processing main body 60 is connected to the cup unit 10 and is able to supply washing water to the washing nozzle unit 21 and suck and store together with the washing water the excrements of the urine sucking space 11 and the feces sucking space 13. The excrement processing main body 60 may allow the user to carry out an excrement process cleanly in cooperation with the cup unit 10.

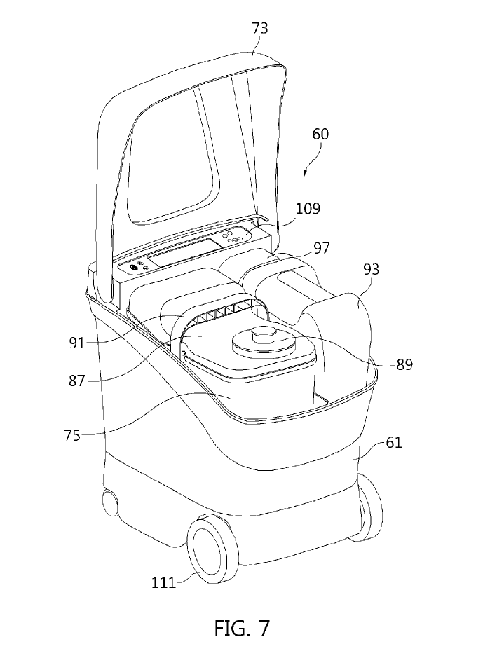

As illustrated in FIG. 7, the case may define the exterior of the excrement processing main body 60. In the inside of the case 61, there are a suction tank 75 and a washing water storing tank 93. The case 61 may be configured in such a way that the inside of the case 61 can be open when the upper cover 73 hinged on the top is opened.

The suction tank 75 and the washing water storing tank 93 may be arranged compact in the insides of the case 61 and the upper cover 73 so as to make the exterior of the excrement processing main body 60 look good. The case 61 may be divided into a suction tank-arranged space 63, a storing tank-arranged space 65, and a suction unit-arranged space 67.

The suction tank 75 is installed detachable in the space 63 wherein the suction tank 75 formed in the inside of the case 61 is arranged. The suction tank 75 is connected through the suction nozzle unit 49 to the cup unit 10 and is able to store the excrement sucked together with the washing water.

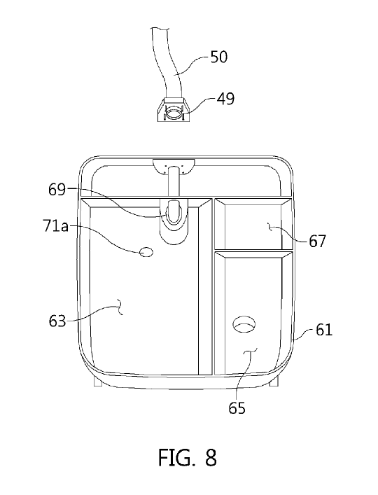

As illustrated in FIG. 8, an excrement collection port 69 is arranged in the space 63 wherein the suction tank 75 is arranged, and is connected with the suction nozzle unit 49, and an excrement inlet port 79 may be formed at the suction tank 75 in such a way to correspond to the excrement collection port 69.

In this case, when the suction tank 75 is inserted into the space where the suction tank 75 is arranged, the excrement inlet port 79 formed in the suction tank 75 will communicate with the excrement collection port 69, so the excrement can be stored in the suction tank 75. At this time, a sealing member (not illustrated) may be arranged at either the excrement collection port 69 or the excrement inlet port 79, thus sealing a communicating portion.

An end of the connection pipe 50 connected to the cup unit 10 can be engaged separable to the excrement processing main body 60, and the suction nozzle unit 49 is protruding from the end of the connection pipe 50 and may be connected with a portion of the excrement collection port 69 of the excrement processing main body 60.

As another example, when the suction tank 75 is inserted into the space 63 wherein the suction tank is disposed, the excrement collection port 69 can position in the inside of the suction tank 75 through the excrement inlet port 79 formed at the suction tank 75, so the excrement can be stored in the suction tank 75.

Non-contact sensors 71a and 71b may be provided at the suction tank 75 and the case 61 so as to check if the case 61 of the suction tank 75 is engaged and check the full presence of the excrement stored in the suction tank 75. The full presence of the excrement may be checked in such a way to detect if the amount of the excrement stored in the inside of the suction tank 75 exceeds a set amount.

Due to the characteristics of the suction tank 75 wherein excrements are stored, it is impossible to install electronic devices in the inside, which are able to detect the amount of excrements. To this end, it is preferred that noncontact sensors 71a and 71b are attached to the portions corresponding to the suction tank 75 and the case 61, thus checking the full presence of the excrements. The noncontact sensors 71a and 71b may be attached multiple in number to the suction tank 75 and the case 61. As for the kinds thereof, for example, magnetic noncontact sensors may be used.

In the present embodiment, the noncontact sensors 71a and 71b are attached to the bottom surface of the suction tank 75 and to the inner surface of the case 61 so as to check if the case 61 of the suction tank 75 has been engaged.

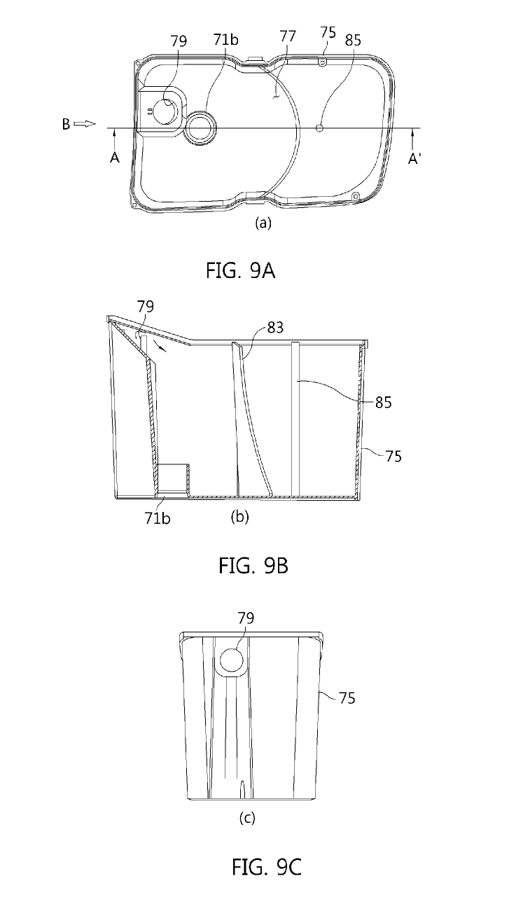

As illustrated in FIGS. 9A to 9C, as for the suction tank 75, the excrements sucked together with the washing water fall from top to bottom and is stored in a space unit 77 in the suction tank 75. The smell of the excrements stored together with the washing water in the space unit 77 does not leak to the outside, and the reverse flow of the excrements can be also prevented since the excrement inlet port 79 positions on the top.

The suction tank 75 may include a partition unit 83 which is able to partition the space unit 77 into two parts. The partition unit 83 is engaged slidable to the groove formed on the inner wall of the suction tank 75, whereupon it can be easily detachable. The other space portion of the suction tank 75 partitioned by the partition unit 83 may include an air discharge pipe 85 to discharge the air expanded due to excrement decomposition.

The air discharge pipe 85 may be formed protrusive in the other space portion partitioned by the partition unit 83. It is preferred that the air discharge pipe 85 is connected to a deodorizing filter (not illustrated) provided at the excrement processing main body 60, thus discharging, to the outside through the deodorizing filter, the air which has been discharged through the air discharge pipe 85.

The deodorizing filter may be formed of a multi-stage deodorizing filter which are made from charcoal, active carbon, platinum, antimicrobial function, non-woven cloth, etc., thus preventing the smell of the excrement from leaking to the outside.

Not illustrated in the drawing, an UV-lamp and a photo catalyst may be installed so as to sterilize the excrements in the suction tank.

As illustrated in FIG. 7, the suction tank 75 my include a main cover 87 for covering the space unit 77. The main cover 87 may further include an assistant cover 89 for opening and closing the space unit 77. It is preferred that a random opening and closing of the assistant cover 89 should be prevented in such a way that it is structured to be closed by a press-in operation. In addition, it is preferred that the suction yank 75 may equip with a handle 91 for the sake of easier transfer and to be easily engaged to the case 61.

The washing water storing tank 93 may be installed detachable in the space 65 wherein the storing tank of the case 61 is disposed. The washing water storing tank 93 may include a water storing space (not illustrated) wherein washing water is stored. A discharge port is provided at one side so as to discharge washing water which has been stored in the water storing space. A residual amount confirmation sensor may be provided at the washing water storing tank 93 so as to confirm the residual amount of the washing water. The residual amount confirmation sensor may be formed of a noncontact sensor.

A water pump 95 and a suction unit 97 may be further provided in the inside of the case 61.

The water pump 95 is provided so as to supply the washing water from the washing water storing tank 93 to the washing nozzle unit 21. The water pump 95 is able to supply the washing water in the washing water storing tank 93 to the washing nozzle unit 21 in accordance with a command from the control unit 107 which will be described later.

The suction unit 97 is provided so as to suck excrement. The suction unit 97 is configured to suck excrement in accordance with a command from the control unit. It is preferred that the suction unit 97 includes a suction motor. The suction unit 97 may further include a fan which rotates by the suction motor and a device, etc. for the sake of air suction.

An instant heater 101 and a supply amount control unit 103 may be further provided in the inside of the case 61.

The instant heater 101 is able to instantly heat the washing water in the course where the washing water in the washing water storing tank 93 is supplied to the washing water nozzle unit 21. As the instant heater 101 operates, the washing water can be supplied in a form of hot water

The supply amount control unit 103 is configured to adjust the amount of washing water which is supplied to the washing water nozzle unit 21 and the cleaning and washing nozzle 29. Through the amount control of the washing water, it is possible to control the supply pressure of the washing water. The supply amount control unit 103 may be a solenoid valve. In the present embodiment, the supply amount control unit 103 may be provided at the sides of the water pump 95 and the assistant pump 105 which will be described later.

The assistant pump 105 may be further provided in the inside of the case 61. The assistant pump 105 is provided so as to supply at a high pressure the washing water in the washing water storing tank 93 to the cleaning and washing nozzle 29.

The water pump 95, the suction motor 99, the instant heater 101, the assistant pump 105, etc. may be disposed in a space which is formed at a lower side of the case 61 and are preferably configured to operate in cooperation with the suction tank 75, the washing water storing tank 93, etc.

There is a control unit 107 configured to command the supply of washing water and the suction of excrement when excrement is detected in the cup unit 10. The control unit 107 may be provided at the cup unit 10 or the excrement processing main body 60. For example, the control unit 107 may be manufactured into a form of a PCB substrate and may be disposed on the bottom surface of the cup unit 10.

The control unit 107 may be configured to command the supply of washing water and the suction of excrement only when a signal from the wearing detection sensor 39 is detected.

In addition, even though a signal from the wearing detection sensor 39 is detected, the control unit 107 may receive detection signals from the noncontact sensors 71a and 71b, thus determining whether or not the suction tank 75 has been engaged to the case 61 or the excrement stored in the suction tank 75 has been full, and then commands the supply of washing water and the suction of excrement.

Even though a signal from the wearing detection sensor 39 is detected, the control unit 107 may receive detection a signal from the residual amount confirmation sensor (not illustrated) and may command the supply of washing water if the amount of the washing water stored in the washing water storing tank 93 exceeds a predetermined amount.

More specifically, the control unit 107 may receive the detection signals from the wearing detection sensor 39, the electric sensor 41, the photo sensor 43 and the noncontact sensors 71a and 71b and may command the supply of washing water and the suction of excrement by determining whether or not the user's hips are contacting close with the close contact unit 33 or whether or not the suction tank 75 has been engaged to the case 61 or whether or not the excrement stored in the suction tank 75 has been full or whether or not the amount of the washing water stored in the washing water storing tank 93 has been full.

When it is determined that the supply of washing water and the suction of excrement have been completed after the control unit 107 commanded the supply of washing water and the suction of excrement, the control unit 107 may allow to supply wind to the drying nozzle unit 31 for the sake of drying. The wind for drying may be supplied to the drying nozzle unit 31 in such a way that the fan 106 sucks external air. Except for the above method, the air for drying may be supplied to the drying nozzle unit 31 by separately providing a device for sucking air.

The control unit 107 may allow to perform washing, suction and drying functions in such a way to receive the information which has been inputted in the display unit 109 which will be described later. The information on the function that the control unit 107 will perform may be displayed on the display unit 109.

The display unit 109 is provided to operate and confirm the operations of the cup unit 10 and the excrement processing main body 60.

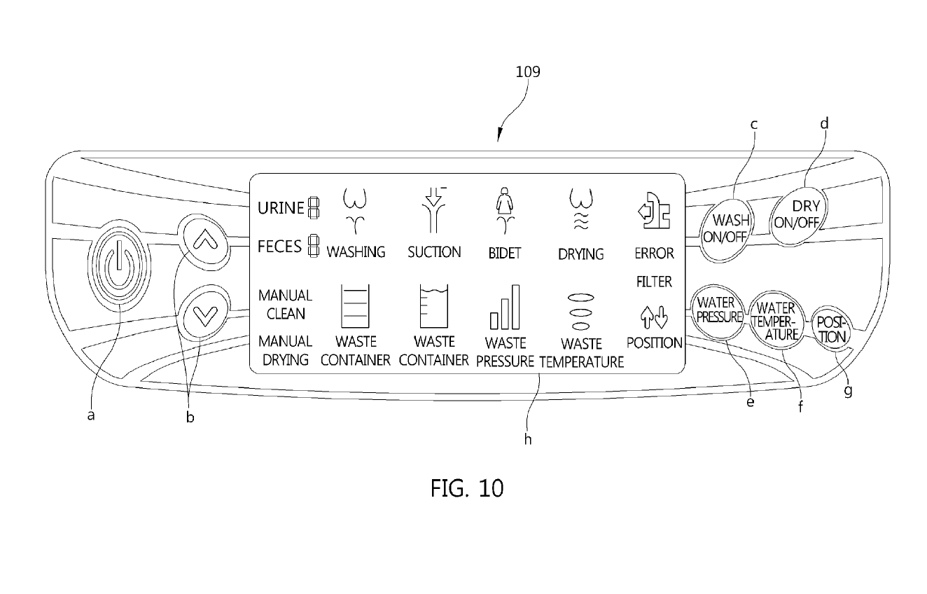

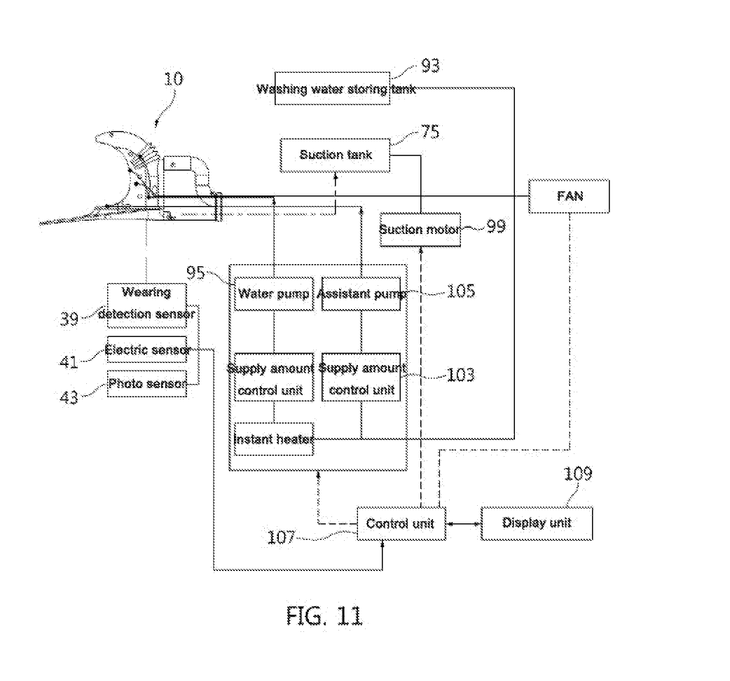

As illustrated in FIG. 10, the display unit 109 may equip with operation buttons to manually or automatically operate the cup unit 10 and the excrement processing main body 60.

The operation button may include, but is not limited to, a power button (a), an up and down control button (b), a washing on/off button (c), a drying on/off button (d), a water pressure button (e), and a position button (g).

The power button (a) has the following function. When the power button (a) is pressed once, power is supplied. When the power button (a) is pressed again, the supply of the power quits. The power button (a) allows to start the operation of the automatic excrement processing device and to quit it.

The up and down control button (b) is a control button for raising or lowering the functions of the washing on/off button (c), the drying on/off button (d), the water pressure button (e), the water temperature button (f) and the position button (g).

The washing on/off button (c) is a button for controlling the operation and stop when more washing is necessary. The drying on/off button (d) is a button for controlling the operation and stop when more drying is necessary. The water pressure button (e) is a button for setting the water pressure of the washing water which is sprayed from the washing nozzle unit. The water temperature button (f) is a button for setting the temperature of washing water to high, middle and low levels.

The position button (g) is a button for setting the water pressure of washing water which is sprayed over the cup unit 10 and the temperature, suction level, etc. of the washing water. More specifically, the position button (g) is a button for setting the water pressure of the washing water for the sake of automatic operation of the excrement processing and the conditions, for example, pressure, the temperature, suction level, etc. of the washing water.

The display unit 109 is able to display on its window at least one among a state on washing, suction, bidet, and drying, an operation error in the cup unit, the exchange of the deodorizing filter, the temperature of washing water, water pressure, the amount of washing water stored in the washing water storing tank 93, the amount of excrement stored in the suction tank 75, and a relative position state between the cup unit 10 and the excrement processing main body 60.

For example, as illustrated in FIG. 10, the display unit 109 may display indication keys corresponding to urine washing time, feces washing time, manual drying, washing, suction, bidet, drying, error, the amount of excrement filled in the suction tank (water container), the amount of washing water in the washing water storing tank, the pressure of washing water (water pressure), the temperature of washing water (water temperature), position, a state on the exchange of deodorizing filter (filter), etc

The urine washing time and feces washing time indication keys show the urine and feces washing time, and if the washing is underway, the remaining time is indicated. The washing, suction, bidet and drying indication keys indicate the currently ongoing functions.

The error indication key is configured to blink if the washing water storing tank 93 or the suction tank 75 has not been engaged at the excrement processing main body 60 or if the amount of the water in the washing water storing tank 93 is below a set level or if the excrement stored in the suction tank 75 is full.

The position indication key is configured to indicate if the cup unit 10 positions higher or lower than the excrement processing main body 60. The control unit 107 may allow to adjust the water pressure of the washing water which is supplied to the washing nozzle unit 21 based on a state where the cup unit 10 positions higher or lower than the excrement processing main body 60.

The water pressure of the washing water may be adjusted by controlling the operation of at least one of the water pump 95, the assistant pump 105 and the supply amount control unit 103. The temperature of the washing water can be adjusted by operating the instant heater.

As illustrated in FIG. 1, the moving wheels 111 are provided so as to allow the excrement processing main body 60 to move freely. The excrement processing main body 60 may equip with a handle portion 113 which may be disposed retractable at one side of the top. The handle portion 113 may be withdrawn to the outside and be used only in the course of movement of the excrement processing main body 60. The excrement processing main body 60 may equip with a power plug 115 for the sake of power supply, and the power plug 115 may be separable from the excrement processing main body.

The operation of the present invention will be described below.

The operation of the cup unit will be first described with reference to FIGS. 2 and 11.

The cup unit 10 may be engaged where the user's excretory organ positions when the upper cup “A” is tilted with respect to the lower cup “B”. Then the cup unit 10 can be engaged closely contacting with the user's excretory organ with the aid of the recovery force of the front portion and the curved shape.

When the cup unit 10 is engaged against the user's excretory organ, the user's anus portion may position where the space 13 into which feces is sucked. In case of male, the genital organ portion may position in the space 11 into which urine is sucked, and in case of female, it may position in the space 13 into which feces is sucked.

At this time, the user's hips may be elastically supported closely contacting with the close contact portion 33, thus having a comfort wearing feeling. In addition, if the user's hips closely contact with the close contact portion 33, the wearing detection sensor 39 disposed at the cup unit 10 will detect it and transmits the detected signal to the control unit 107.

In this state, when the user pees or excavates feces, the electric sensor 41 provided at the cup unit 10 and the photo sensor 43 detect the urine or feces, and the detected signal is transmitted to the control unit 107.

The control unit 107 may receive signals from the wearing detection sensor 39, the electric sensor 41 and the photo sensor 43 and may further receive signals from the suction tank 75 and the noncontact sensors 71a and 71b provided at the washing water tank 93 and may determine if the suction tank 75 and the washing water storing tank 93 have been engaged at the excrement processing main body 60, if the suction tank 75 is full and if the amount of the washing water stored in the washing water storing tank 93 exceeds a set amount.

As a result of the determination, if it is determined that the suction tank 75 and the washing water storing tank 93 have been well engaged at the excrement processing main body 60, and the suction tank 75 has not been full, and the amount of the washing water stored in the washing water storing tank 93 has exceeded a set amount, and the user has peed or evacuated feces, the control unit 107 commands the suction of excrement and the washing of excretory organ.

For example, as a result of the determination by the control unit 107, if the excrement is urine, the control unit 107 may allow to wash and at the same time suck the washing water and urine with the aid of suction force of air by operating the water pump 95 and the suction motor 99, and the urine sucked together with the washing water is transferred through the suction nozzle unit 49 to the excrement processing main body 60 and is stored in the suction tank 75.

As a result of the determination by the control unit 107, if the excrement is feces, the control unit 107 may allow to process feces in such a way that the suction force of air and washing water cooperate, by simultaneously operating the suction motor 99, the water pump 95 and the assistant pump 105. Namely, when the user excavates feces, a washing function may be performed, wherein the anus can be washed just as the feces is sucked by the suction force of air, and the washing water is sprayed from the washing nozzle 29 which is able to push the excrement in the direction of the suction nozzle unit 49.

The washing water sprayed from the washing nozzle 29 pushes with a high pressure the excrement toward the suction nozzle unit 49, whereupon the excrement can be easily sucked into the suction nozzle unit 49, and at the same time the washing on the bottom surface of the space 13 into which the feces is sucked can be performed in a 3D direction. To this end, the bottom surface of the space 13 into which the suction is sucked can be washed clean. In addition, the excrement sucked into the suction nozzle unit 49 is transferred to the excrement processing main body 60 together with the washing water and is stored in the suction tank 75.

At this time, the air hole 45 formed at the cup unit 10 is configured to prevent hernia of a user by avoiding to form a vacuum state in the space 13 into which feces is sucked, which may occur due to the suction pressure of air.

If the close contact state between the hips and the close contact unit 33 is removed in the course of suction of excrement, the wearing detection sensor 39 detects it and transmits a signal to the control unit 107. The control unit 107 which has received a signal of the wearing detection sensor 39 will stop the supply of the washing water by stopping the operations of the water pump 95 and the assistant pump 105, which is performed so as to avoid the wetting of sheet by the washing water in such a way to stop the supply of washing water which is used to wash the anus when the close contact state between the hips and the close contact unit 33 is removed.

This cup unit 10 is able to carry out a suction function of excrement just as the excrement is excavated, whereupon the feces and urine can be directly sucked not staying in the cup unit. For this reason, it is possible to prevent any exposure to disease and prevent skin pressure sore.

In addition, since the cup unit 10 is able to provide a comfort wearing feeling since the close contact unit can be substituted based on the sizes of the user's hips.

Next, the operation of the excrement processing main body will be described.

When the cup unit 10 is disposed at a portion where the user's excretory organ positions, the hip portions where the anus of the user's excretory organ positions may contact close with the close contact unit 33 of the cup unit 10.

At this time, since the cup unit 10 is made of an ABS material, and only the close contact unit 33 contacting with the user's hips is made of a silicon material, a sense of wearing is good, and the detection power of the sensor unit is high, thus accurately detecting the feces and urine.

In this state, when the power button (a) of the excrement processing main body 60 is pressed, the cup unit 10 and the excrement processing main body 60 will be ready for operation.

Thereafter, when the user excavates excrement in the cup unit 10, the control unit 107 which has received signals from the electric sensor 41 provided at the cup unit 10 and the photo sensor 43 will determine if the excrement is urine or feces and will command the suction and washing of excrement.

At this time, if it is determined that the user has excavated excrement in a state where the suction tank 75 and the washing water tank 93 have been engaged on the excrement processing main body 60, the suction tank 75 has not been full, the amount of the washing water stored in the washing water storing tank 93 has exceeded a set amount, and the signal from the wearing detection sensor 39 has been detected, the control unit 107 will command the suction of excrement and the washing of the excretory organ by determining if the excrement is urine or feces.

If it is determined that the excrement is urine, the control unit 107 may allow to operate the water pump 95 and the suction motor 99, and if it is determined that the excrement is feces, the control unit 107 may allow to simultaneously operate the water pump 95 and the suction motor 99, and the assistant pump 105.

If it is determined that the excrement is both the urine and feces, the control unit 107 may allow to simultaneously operate the water pump 95 and the suction motor 99, and the assistant pump 105. The simultaneous operations of the water pump 95, the suction motor 99 and the assistant pump 105 may allow to simultaneously carry out the washing of excretory organ and the suction of excrement, thus obtaining a clean processing of the excrement by pushing the excrement in the direction of the suction nozzle unit 49.

The path where the washing water is supplied by the operations of the water pump 95 and the assistant pump 105 can be confirmed the solid line and the dotted line in FIG. 11.

In addition, when the user needs to adjust the temperature of the washing water and the water pressure, the user can press the operation button. To this end, the control unit 107 may allow to command the operations of the instant heater 101 and the supply amount control unit 103, so that the washing water which will be supplied to the washing nozzle unit 21 can be instantly heated or the water pressure can be adjusted.

Meanwhile, the washing water sprayed over the cup unit through the washing nozzle unit 21 will wash the user's anus or genital organ portion and will be inputted through the suction nozzle unit 49 into the space unit 77 of suction tank 75 together with the excrement with the aid of suction of air of the suction motor 99 which is able to carry out the suction of excrement.

The excrement and washing water sucked through the suction nozzle unit 49 fall down from top to bottom through the excrement inlet port 79 of the suction tank 75 and are stored in the space unit 77 in the suction tank 75. To this end, the smell of the excrement stored in the suction tank 75 does not leak outward. In addition, as for the suction tank 75, since the excrement inlet port 79 positions on the top, a problem does not occur, wherein the excrement flows reversely through the suction nozzle unit 49.

The suction tank 75 is divided into the partition unit 83 and the space unit 77, and the excrement is stored only in the space unit 77, thus coping with the expansion which occurs due to the decomposition of the excrement.

The air expanded due to the decomposition of the excrement may discharge to the outside through the air discharge pipe 85 connected with the deodorizing pipe 85, whereupon the smell of the excrement does not leak outward.

After the excrement evacuation has been completed, and the suction of the excrement has been completed, a drying function with respect to the excretory organ will be performed, wherein wind or hot water may be discharged through the drying nozzle unit 31. When the drying function with respect to the excretory organ has been completed, the cup unit 10 is separated from the user, and the power button (a) provided on the top of the excrement processing main unit 60 is pressed, and then the operable state of the excrement processing main body 60 is finished.

For the sake of cleaning the suction tank 75, the upper cover 73 of the excrement processing main body 60 is opened, and then the suction tank 75 is withdrawn. The withdrawing of the suction tank 75 may be formed in such a way that the user grabs the handle 91 engaged to the top of the suction tank and separates the suction tank 75 from the excrement processing main body 60.

Next, the main cover 87 or the assistant cover 89 of the suction tank 75 is opened, and the excrement being inside is empted, and the partition unit 83 is separated, and the space unit 77 is cleaned. At this time, since the partition unit 83 can be separated slidable from the suction tank 75, the cleaning in the space unit 77 may be more easily carried out.

The supplement of the washing water into the washing water storing tank 93 may be carried out in a similar method with the cleaning method in the suction tank 75 which has been described above.

Since the excrement processing main body 60 may indicate to the outside at least one among a state on washing, suction, bidet, a cup unit operation error state, an exchange state of a deodorizing filter, a washing water temperature, the amount of the washing water stored in the washing water storing tank 93, and the amount of excrement stored in the suction tank and a relative position state between the cup unit and the excrement processing main body 60, it is easy to cope with any situation in such a way to confirm the operation and maintenance states of the excrement processing main body 60.

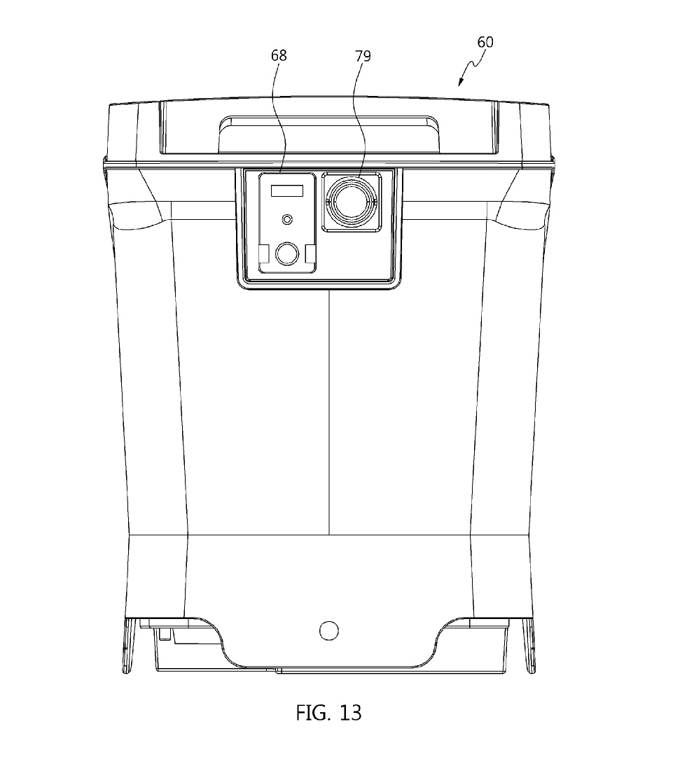

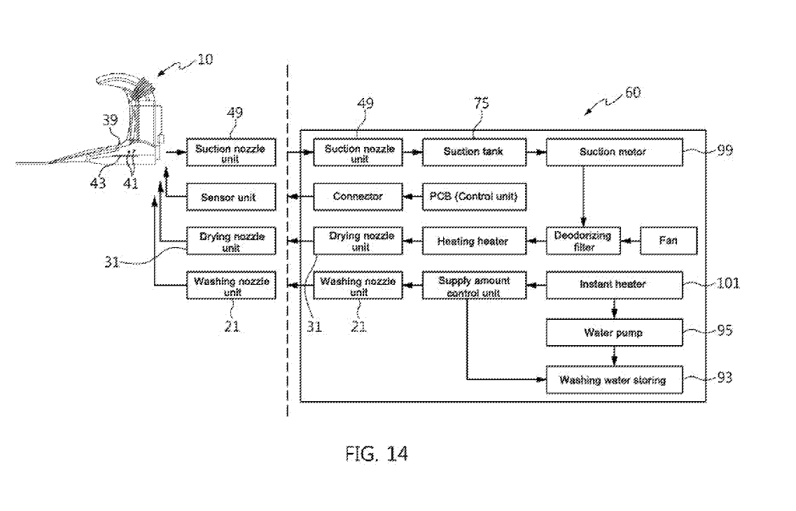

FIGS. 12 and 13 show the backsides for connecting the cup unit and the excrement processing main unit trough the connection pipe according to another exemplary embodiment.

As illustrated in FIG. 12, on the backside of the cup unit 10, the discharge passage 47 and the coupling unit 48 are formed. As illustrated in FIG. 13, on the backside of the excrement processing main body 60, the excrement inlet port 79 and the coupling unit engaging unit 68 are formed.

To this end, the suction nozzle unit 49 can be connected solely in the connection pipe 50 and with the excrement processing main body 60 in such a way that one side of the suction nozzle unit 49 is connected with the discharge passage 47 of the cup unit 10 and the other side thereof is connected with the excrement inlet port 79.

In addition, the washing nozzle unit 21, the drying nozzle unit 31 and the sensor unit may be constituted together, so one side thereof can be connected in the connection pipe 50 and with the coupling unit 48, and the other side thereof can be connected with the coupling unit engaging unit 68 of the excrement processing main body 60.

To this end, the engaging and disengaging of the cup unit 10 and the connection pipe 50, and the connection pipe 50 and the excrement processing main body 60 are easy, and the cleaning of the cup unit 10, the suction nozzle unit 49 in the connection pipe 50 and the washing nozzle unit 21, etc. is easy.

FIG. 14 shows an operation principle of the automatic excrement processing device according to another exemplary embodiment of the present invention. The another exemplary embodiment in FIG. 14 describes the most basic operation principle wherein the suction and washing of the excrement are performed.

As illustrated in FIG. 14, the sensor unit, the washing nozzle unit 21, the suction nozzle unit 49 and the drying nozzle unit 31 which are provided in the cup unit 10 are connected with the excrement processing main body 60.

The sensor unit may includes, but is not limited to, a wearing detection sensor 39, an electric sensor 41 and a photo sensor 43 and is connected through a connector with the excrement processing main body 60 and is configured to transfer a detection signal to the PCB (control unit).

The washing nozzle unit 21 is connected with the washing water storing tank 93. The washing water of the washing water storing tank 93 can be heated by the instant heater 101 while it is supplied to the washing nozzle unit 21, and the supply amount control unit 103 can adjust the amount of washing water which is supplied to the washing nozzle unit 21. The washing nozzle unit 21 may include, but is not limited to, a men's washing nozzle 23, a women's washing nozzle 25, an anus washing nozzle 27, and a cleaning washing nozzle 29. Since the men's washing nozzle 23, the women's washing nozzle 25, the anus washing nozzle 27, and the cleaning washing nozzle 29 operate together, the excrement processing can be cleanly carried out.

The washing water storing tank 93 may include a main water container and an assistant water container. The capacity of the main water container may be 6.5 L, and the capacity of the assistant water container may be 1.5 L. The washing nozzle unit 21 may have a diameter of 5 mm.

The suction nozzle unit 49 is connected with the suction tank 75. Since the suction motor 99 operates, the suction of the excrement and washing can be carried out through the suction nozzle unit 49. The excrement and washing water sucked through the suction nozzle unit 49 are inputted into the suction tank 75 and are stored. The capacity of the suction tan may be 7500 cc.

The drying nozzle unit 31 is connected with a fan. Since the fan operates, external air can be inputted. A deodorizing filter having deodorizing, air freshening and sterilization functions may be provided where the external air is inputted, thus preventing impurities from being inputted together when the external air is inputted.

The air which has passed through the deodorizing filer may be heated while passing through the heating heater, whereupon the hot air can be transferred to the user's hips through the drying nozzle unit 31. The diameter of the drying nozzle unit 31 may be 16 mm.

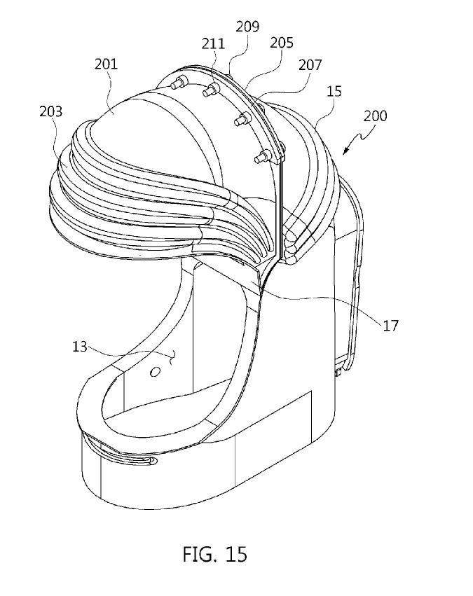

As still further another exemplary embodiment, FIG. 15 shows a cup unit according to another exemplary embodiment of the present invention.

The cup unit illustrated in FIG. 15 has a difference in the way that the cup unit includes a contractible unit as compared with the another exemplary embodiment of the present invention.

More specifically, the cup unit 200 may include a contractible unit 201 which is fixedly connected through an engaging member with the side of the space 11 which receives urine, so that the front side of the contractible unit 201 can be formed curved and contractible. At least one portion of the contractible unit 201 may be formed in a Zabara type 203, so that the front side of the space 11 into which the urine in the cup unit 200 is sucked can be contracted toward the rear side within a predetermined range.

As for the cup unit 200 according to another exemplary embodiment of the present invention, the upper cup can be tilted with respect to the lower cup with the aid of cooperation of the contractible unit 201 and the wrinkle unit 15, and the curve shape at the front side of the cup unit 200 may be made more gentle. That the curved shape at the front side of the cup unit 200 is made more gentle may allow for the cup unit 200 to be commonly worn by the users who have various body sizes while improving a wearing comfort.

Flanges 205 and 207 which are protruding facing each other may be formed at the portion where the contractible unit 201 is connected with the cup unit 200, and each of the flanges 205 and 207 has an engaging hole. The engaging member is engaged to the engaging hole, and the contractible unit 201 is fixedly connected with the cup unit 200.

The engaging member may be an engaging bolt 209 which is fixed at the engaging nut 209 which is engaged passing through the engaging hole and at an end portion of the engaging nut 211 which passes through the engaging hole, thus fixing a state where the engaging nut 211 is engaged to the engaging hole.

The cup unit 200 according to another exemplary embodiment of the present invention may have an advantage in more improving the wearing comfort of the cup unit by the user as compared with the exemplary embodiment.

The cup unit 200 according to the above another exemplary embodiment of the present invention is same as the exemplary embodiment except for the contractible unit 201, whereupon the description on the same components will be omitted.

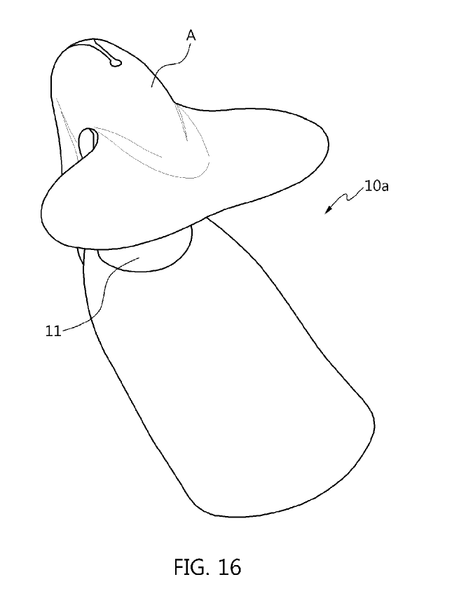

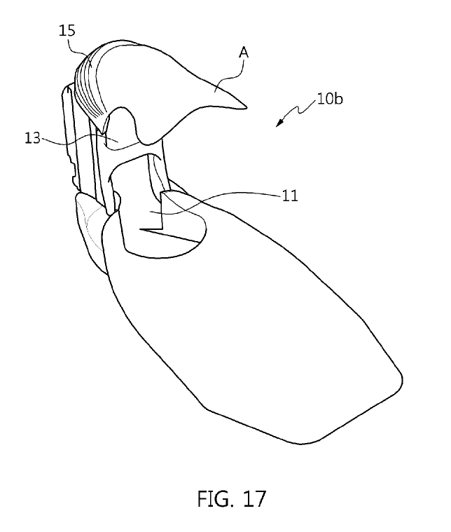

As further another exemplary embodiment of the present invention, FIGS. 16 and 17 show the cup unit according to further another exemplary embodiment of the present invention.

The cup unit 10a illustrated in FIG. 16 has a difference in the way that the shape of the end of the upper cup “A” of the cup unit 10a is formed in a shape of a wing (dolphin shape) as compared with the exemplary embodiment of the present invention.

The wing-shaped (dolphin) end of the upper cup “A” is able to widely cover the front side of the user's excretory organ, thus providing more comfortable wearing.

As further another embodiment of the present invention, FIG. 17 shows a cup unit according to further another embodiment of the present invention.

The cup unit 10b illustrated in FIG. 17 has a difference in the way that the shape of the end portion of the upper cup “A” of the cup unit 10a is a wing shape having an inwardly concave curve line as compared with the exemplary embodiment of the present invention.

The wing-shaped end portion of the upper cup “A” can contact close with the front side of the user's excretory organ when the user wears the cup unit 10b, thus enhancing a comfort wearing and a close contact power.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. An automatic excrement processing device, comprising:
a cup unit which is worn at a portion corresponding to a user's excretory organ and is divided into a space for sucking urine and a space for sucking feces;
an excrement processing main body which is connected with the cup unit and is configured to suck and store the excrement which is excavated into the cup unit;
a sensor unit which is provided in the cup unit, is connected with the excrement processing main body, and is able to detect the excrement;
a washing nozzle unit which is configured to carry out a washing function;
a suction nozzle unit which is able to suck the excrement; and
a drying nozzle unit which is able to carry out a drying function,
wherein when the user excavates excrement, the excrement can be automatically detected, and a washing, suction, bidet and drying can be carried out.

2. The device of claim 1, wherein the cup unit comprises an upper cup having a space for sucking urine and a lower cup having a space for sucking excrement, and the front side of the cup unit is formed in a C-shaped curve, and the cup unit comprises a wrinkle unit at its backside in order for the upper cup to tilt with respect to the lower cup.

3. The device of claim 1, wherein the cup unit is made of an ABS material, and a portion contacting with a user is covered with a silicon material.

4. The device of claim 1, wherein the cup unit comprises an air hole which forms an air flow path at its side surface while allowing to receive air through the rear end of the cup unit so as to prevent any vacuum in the space for sucking urine and the space for feces.

5. The device of claim 1, wherein the washing nozzle unit comprises:
a men's washing nozzle which is exposed into the space for sucking urine;
a women's washing nozzle which is expose into the space for sucking feces;
an anus washing nozzle which is installed on the bottom surface of the space for sucking feces and is exposed into the space for sucking urine and is able to wash the front and rear sides of the anus; and
a cleaning and washing nozzle which is able to push the excrement in the direction of the suction nozzle unit in such a way to spray washing water toward the bottom surface of the space for sucking feces.

6. The device of claim 5, wherein the air discharged through the drying nozzle unit into the cup unit is inputted into the backside of the cup unit and is sprayed again over the cup unit through the cleaning and washing nozzle.

7. The device of claim 1, wherein the sensor unit comprises:
a wearing detection sensor which is installed at the cup unit and is able to detect if user's hips contact close with the cup unit;
an electric sensor which is installed at an edge of the bottom surface of the cup unit and is able to detect urine and feces; and
a photo sensor which is installed on the bottom surface of the cup unit and is able to detect feces.

8. The device of claim 1, wherein the cup unit is connected through a connection pipe with the excrement processing main body, and in the connection pipe, the suction nozzle unit is solely connected with the excrement processing main body, and the washing nozzle unit, the drying nozzle unit and the sensor unit are connected through a coupling unit, which is provided together, with the excrement processing main body, whereupon the cup unit and the connection pipe, and the connection pipe and the excrement processing main body can be easily engaged or disengaged.

9. The device of claim 8, wherein the suction nozzle unit comprises an automatic valve which is opened when the excrement is detected in the cup unit.

10. The device of claim 1, wherein the excrement processing main body comprises:
a case which defines an exterior of the device;
a washing water storing tank which is provided detachable in the case and is configured to store washing water and is connected with the washing nozzle unit;
a suction tank which is provided detachable in the case and is configured to store the excrement which has been sucked together with the washing water and is connected with the suction nozzle unit;
a deodorizing filter which is provided so as to remove smell from the excrement in the suction tank; and
a UV-lamp and photo catalyst which are provided or the sake of sterilization.

11. The device of claim 10, further comprising:
a control unit which is configured to command a washing, suction, bidet and drying when any excrement is detected in the cup unit;
a water pump which is configured to supply the washing water in the washing water storing tank to the washing nozzle unit in accordance with a command from the control unit;
a suction motor which is able to carry out a suction of the excrement in accordance with a command from the control unit; and
a fan which is configured to supply air to the drying nozzle unit in accordance with a command from the control unit.

12. The device of claim 11, further comprising:
an deodorizing filter which is able to remove any impurities while the air is supplied to the drying nozzle unit; and
a heating heater which is able to heat the air which is being supplied to the drying nozzle unit.

13. The device of claim 10, further comprising:
an instant heater which is configured to instantly heat the washing water while the washing water in the washing water storing tank is being transferred to the washing nozzle unit; and
a supply amount control unit which is configured to adjust the amount of the washing water which is being supplied to the washing water nozzle unit.

14. The device of claim 11, wherein the cup unit comprises:
a wearing detection sensor which is configured to detect if the user's hips contact close with the cup unit, wherein the control unit commands a washing, suction, bidet, and drying only when the wearing control unit receives a detection signal from the wearing detection sensor.

15. The device of claim 11, wherein the suction tank and the case comprise a noncontact sensor which is configured to check if the suction tank has been engaged to the case and the full presence of the excrement in the suction tank, and by receiving a detection signal of the noncontact sensor, the control unit is able to determine if the suction tank has been engaged to the case and if the excrement stored in the suction tank has been full, and the control unit commands a washing, suction, bidet and drying.

16. The device of claim 11, wherein the washing water storing tank comprises a residual amount confirmation sensor so as to confirm the residual amount of the washing water, and if the control unit determines that the washing water exceeds a set amount by receiving a detection signal from the residual amount confirmation sensor, the control unit commands a washing, suction, bidet and drying.

17. The device of claim 11, further comprising a display unit for operating and confirming the operations of the cup unit and the excrement processing main body, and the display unit comprises a power button and washing, drying, water temperature control and water pressure control buttons and is configured to display to the outside at least one among a state of washing, suction, bidet or drying, a cup unit operation error state, an exchange of the deodorizing filter, the temperature of the washing water, a water pressure, the amount of the washing water stored in the washing water storing tank, the amount of the excrement stored in the suction tank and a relative position state between the cup unit and the excrement processing main body.
